(19) United States
(12) Patent Application Publication
(10) Pub. No.: US 2024/0390540 A1
(43) Pub. Date: Nov. 28, 2024

(54) ORTHOPAEDIC SURGICAL INSTRUMENT SYSTEM INCLUDING AN INSTRUMENT STERILIZATION TRAY HAVING A MASKING LAYER

(71) Applicant: DEPUY IRELAND UNLIMITED COMPANY, RINGASKIDDY (IE)

(72) Inventor: Alec P. Birkbeck, Leeds (GB)

(21) Appl. No.: 18/202,518

(22) Filed: May 26, 2023

(51) Int. Cl. A61L 2/24; A61B 50/33

(52) U.S. Cl. CPC A61L 2/24; A61B 50/33; A61L 2202/24

(57) ABSTRACT

An orthopaedic surgical instrument system includes a sterilization tray for storage of orthopaedic surgical instruments during sterilization and transport thereof. The sterilization tray has a masking layer disposed on the upper surface of its bottom wall.

### CROSS REFERENCE

Cross reference is made to copending U.S. Design patent application Ser. No. ______ entitled “ORTHOPAEDIC SURGICAL INSTRUMENT WITH SURFACE ORNAMENTATION” (Attorney Docket No. 265280-381493, DEP7154USDP1), which is assigned to the same assignee as the present application and filed concurrently herewith.

### TECHNICAL FIELD

The present disclosure relates generally to orthopaedic surgical instruments for use in the performance of an orthopaedic surgical procedure, and more particularly to a sterilization tray for use in the storage and transport of orthopaedic surgical instruments.

### BACKGROUND

During an orthopaedic surgery such as an orthopaedic trauma procedure or joint replacement procedure, the orthopaedic surgeon typically uses a variety of different orthopaedic surgical instruments such as, for example, handles, drills, reamers, drill guides, cutting blocks, prosthetic trials, and other surgical instruments to prepare the patient's bones to receive an orthopaedic implant such as a fracture plate or joint prosthesis. Such surgical instruments may be constructed of either metal or polymer materials and are generally designed to be reusable. After use, reusable surgical instruments are kitted into sterilization trays, sterilized, and returned to the operating room for use in a subsequent procedure.

Some sterilization trays rely on highly visible color markers to denote certain aspects of the instruments contained in the tray such as the size and functionality of the instruments. Typically this is carried out with solid color printing onto the bottom wall and instrument retainers within the tray. During the cleaning and sterilization process, even though the instruments have been thoroughly and safely sterilized, water stains and water marks may appear on the bottom wall of the tray. While these water stains and water marks occur on most, if not all, sterilization trays, they are much more visible on painted sterilization trays.

### SUMMARY

According to one aspect of the disclosure, a surgical instrument sterilization tray includes a bottom wall having an upper surface and a lower surface, and a plurality of sidewalls extending upwardly from the bottom wall so as to cooperate with the bottom wall to define an instrument storage surface area. A plurality of instrument retainers are secured to the bottom wall and are configured to retain orthopaedic surgical instruments during sterilization and transport thereof. A masking layer is disposed on the upper surface of the bottom wall within the instrument storage area. The masking layer defines a disruptive pattern that includes a plurality of connected common geometric shapes. The masking layer has a pattern coverage ratio of at least 50:50.

In an embodiment, the masking layer is printed onto the upper surface of the bottom wall. In another embodiment, the masking layer is etched into the upper surface of the bottom wall.

In one embodiment, the masking layer has a pattern coverage ratio of at least 60:40.

The bottom wall may have a plurality of fluid holes formed therein.

In an embodiment, the common geometric shape is a circle. In another embodiment, the common geometric shape is a hexagon.

The bottom wall and the plurality of sidewalls may be constructed of anodized aluminum.

According to another aspect, an orthopaedic surgical instrument system includes a surgical instrument sterilization tray constructed of anodized aluminum. The sterilization tray includes a bottom wall having an upper surface and a lower surface, and a plurality of sidewalls extending upwardly from the bottom wall so as to cooperate with the bottom wall to define an instrument storage surface area. The orthopaedic surgical instrument system also includes a plurality of instrument retainers that are secured to the bottom wall of the sterilization tray and configured to retain orthopaedic surgical instruments during sterilization and transport thereof. A masking layer is disposed on the upper surface of the bottom wall of the sterilization tray within the instrument storage area. The masking layer defines a disruptive pattern that includes a plurality of connected common geometric shapes. The masking layer has a pattern coverage ratio of at least 50:50.

In an embodiment, the masking layer is printed onto the upper surface of the bottom wall. In another embodiment, the masking layer is etched into the upper surface of the bottom wall.

In one embodiment, the masking layer has a pattern coverage ratio of at least 60:40.

The bottom wall may have a plurality of fluid holes formed therein.

In an embodiment, the common geometric shape is a circle. In another embodiment, the common geometric shape is a hexagon.

According to another aspect, an orthopaedic surgical instrument system includes a surgical instrument sterilization tray constructed of anodized aluminum. The sterilization tray includes a bottom wall having an upper surface, a lower surface, and a plurality of fluid holes formed therein. Each of the plurality of fluid holes extends from the upper surface to the lower surface. The sterilization tray also includes a plurality of sidewalls extending upwardly from the bottom wall so as to cooperate with the bottom wall to define an instrument storage surface area. The orthopaedic surgical instrument system also includes a plurality of instrument retainers that are secured to the bottom wall of the sterilization tray and configured to retain orthopaedic surgical instruments during sterilization and transport thereof. A masking layer is disposed on the upper surface of the bottom wall of the sterilization tray within the instrument storage area. The masking layer defines a disruptive pattern that includes a plurality of connected common geometric shapes. The masking layer has a pattern coverage ratio of at least 60:40.

In an embodiment, the masking layer is printed onto the upper surface of the bottom wall. In another embodiment, the masking layer is etched into the upper surface of the bottom wall.

In an embodiment, the common geometric shape is a circle. In another embodiment, the common geometric shape is a hexagon.

### BRIEF DESCRIPTION OF THE DRAWINGS

The detailed description particularly refers to the following figures, in which:

FIG. 1 is an elevation view of an exemplary embodiment of an orthopaedic surgical instrument system;

FIG. 2 is an enlarged elevation view showing the masking layer of the sterilization tray in greater detail, with FIG. 2 being taken from FIG. 1 as indicated by the square-shaped area; and

FIG. 3 is a view similar to FIG. 2, but showing another embodiment of the masking layer.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and orthopaedic surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

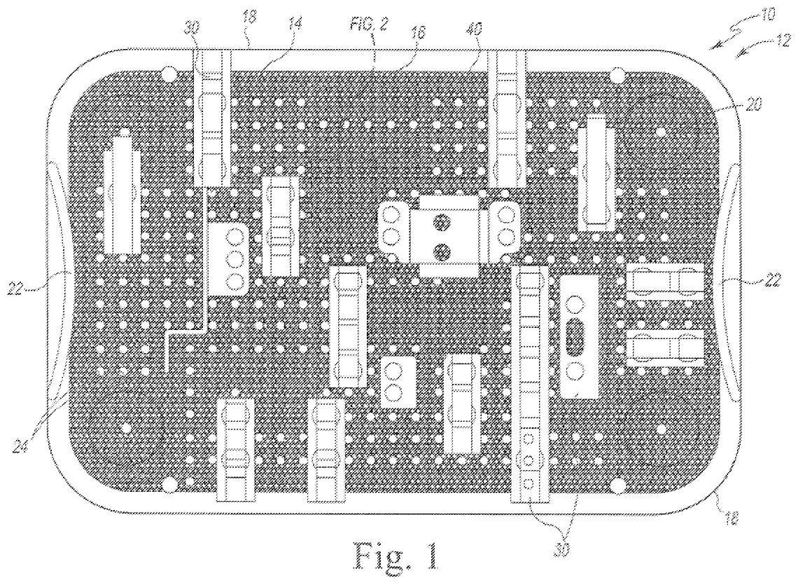

Referring now to FIG. 1, an orthopaedic surgical instrument system 10 is shown. The surgical instrument system 10 includes a sterilization tray 12 used to store a number of orthopaedic surgical instruments (not shown) during sterilization, storage, and transport thereof. What is meant herein by the term “orthopaedic surgical instrument” or “orthopaedic surgical instrument system” is a surgical tool for use by a surgeon in performing an orthopaedic surgical procedure. As such, it should be appreciated that, as used herein, the terms “orthopaedic surgical instrument” and “orthopaedic surgical instrument system” are distinct from orthopaedic implants or prostheses that are surgically implanted in the body of the patient.

As can be seen in FIG. 1, the sterilization tray 12 includes a base or bottom wall 14 that includes an upper surface 16 and an opposite, lower surface. The sterilization tray 12 is generally rectangular in shape with rounded corners. A plurality of sidewalls 18 extend upwardly from the tray's bottom wall 14. In doing so, the sidewalls 18 cooperate with the bottom wall 14 to define an instrument storage surface area 20. A pair of handles 22 are positioned on opposite sides of the sterilization tray 12 for use by surgical staff when carrying the tray 12.

As can be seen in FIG. 1, the bottom wall 14 of the sterilization tray 12 has a plurality of fluid holes 24 formed therein. The fluid holes 24 extend all the way through the bottom wall 14 (i.e., from the bottom wall's upper surface 16 to its opposite, lower surface). In such a way, the fluid holes 24 allow for the ingress and egress of sterilization fluid (e.g., water) into, and out of, the instrument storage surface area 20, thus allowing for sterilization of the orthopaedic surgical instruments positioned therein. It should be appreciated that fluid holes may also be formed in the sidewalls 18 of the sterilization tray 12.

The sterilization tray 12 has a number of instrument retainers 30 secured to its bottom wall 14. The instrument retainers 30 are configured to retain various differently-shaped orthopaedic surgical instruments during sterilization, storage, and transport of the surgical instrument system 10. The instrument retainers 30 may be configured as brackets, connectors, cradles, or any other type of mechanism to retain orthopaedic surgical instruments in a desired location and orientation. It should be appreciated that the instrument retainers 30 could also take the form of specifically-shaped recesses or cavities formed in the instrument sterilization tray 12 into which orthopaedic surgical instruments could be inserted and retained.

In the illustrative embodiment, the sterilization tray 12 is formed from a metallic material such as, for example, anodized aluminum. In particular, the bottom wall 14 and the sidewalls 18 form a metallic assembly assembled from two or more separate components. Alternately, the bottom wall 14 and the sidewalls 18 may take the form of a single monolithic metallic component. The sterilization tray 12 may be formed by conventional machining techniques, or alternatively, by the use of 3-D printing technology. In the case of 3-D printing, the sterilization tray 12 is formed in a layer-by-layer fashion.

As can be seen in FIG. 1, the upper surface 16 of the bottom wall 14 within the instrument storage area 20 has a masking layer 40 disposed thereon. The masking layer 40 masks the presence of any water spots and/or water stains that occasionally accumulate on the upper surface 16 of the bottom wall 14 during the sterilization process. To do so, the masking layer 40 is structured as a disruptive pattern that includes a plurality of connected common geometric shapes. As used herein, the term “disruptive pattern” is defined to mean a form of camouflage that functions by breaking up the outlines of the structure to be camouflaged by use of a strongly contrasting pattern. In the case at hand, the disruptive pattern is used to break up the outlines of the water mark or water stain. As shown in FIGS. 1-3, in the illustrative embodiments described herein, the masking layer's disruptive pattern is sufficiently irregular (by virtue of being embodied as an offset array of four slightly different geometric shapes) to cause visual disruption.

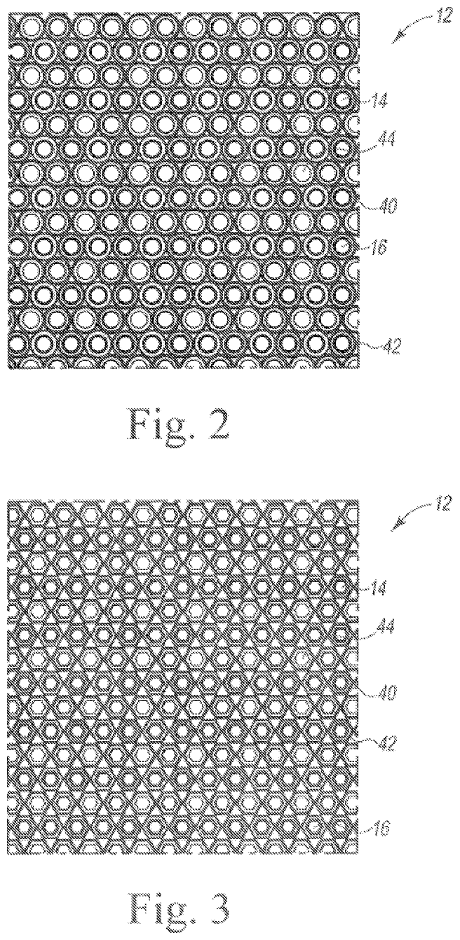

As noted above, the masking layer's disruptive pattern is embodied with a common geometric shape. What is meant herein by the term “common geometric shape” is that all of the geometric shapes of the disruptive pattern are of the same type. For example, as shown in FIGS. 1 and 2, all of the geometric shapes of the masking layer's disruptive pattern are circles. It should be appreciated that the masking layer's disruptive pattern may be embodied with other common geometric shapes. For example, as shown in FIG. 3, all of the geometric shapes of the masking layer's disruptive pattern may be embodied as hexagons. Further examples of geometric shapes that may be utilized in the masking layer's disruptive pattern include triangles, squares, and other regular polygons.

As noted above, the common geometric shapes of the masking layer's disruptive pattern are connected. What is meant herein by the term “connected” as it relates to the common geometric shapes of the masking layer's disruptive pattern is that the outer edges of adjacent geometric shapes contact one another. Doing so creates a uniform connectedness within the disruptive pattern. In Gestalt principles, “Uniform Connectedness” refers to the “grouping effect” in which connected objects that are similar are perceived to be visually connected.

In the illustrative embodiments described herein, each of the connected geometric shapes of the masking layer's disruptive pattern have an outer periphery or “footprint” of the same size. For example, the outer periphery of each of the connected circles has the same circumference even though the makeup of the unconnected circles (i.e., the circles within the connected circles) of a given connected circle may be different than that of an adjacent connected circle (e.g., line weight and shade).

As can be seen in FIGS. 2 and 3, the masking layer's disruptive pattern is relatively dense and therefore covers much of the upper surface 16 of the bottom wall 14. In particular, the masking layer's disruptive pattern has a relatively high pattern coverage ratio. As used herein, the term “pattern coverage ratio” refers to the ratio of the aggregate surface area of the applied pattern relative to the aggregate surface area of the background. In other words, within a given surface area of the tray's bottom wall 14 upon which the masking layer 40 is disposed, some of the surface area will be covered by the applied pattern (that is, the portions of FIGS. 2 and 3 shown in dark lines as referenced by reference numeral 42) and some of the surface area will be not be covered (that is, the portions of FIGS. 2 and 3 shown as white spaces as referenced by reference numeral 44). The uncovered portion of the surface area is the background (in which the bare metal is visible). Within a given surface area of the tray's bottom wall 14 upon which the masking layer 40 is disposed, the “pattern coverage ratio” refers to the ratio of the aggregate surface area of the tray's bottom wall 14 in which the pattern is applied (e.g., printed) relative to the aggregate surface area of the tray's bottom wall 14 which is uncovered—i.e., the background. In an illustrative embodiment, the pattern coverage ratio of the masking layer's disruptive pattern is 50:50. In another embodiment, more of the surface area is covered than uncovered and the pattern coverage ratio of the masking layer's disruptive pattern is 60:40.

The masking layer 40 may be disposed on the upper surface 16 of the bottom wall 14 in a number of different manners. For example, the masking layer 40 may be printed on the bottom wall 14, such as by the use of sublimation printing. The masking layer 40 may be painted on the bottom wall 14. In another embodiment, the masking layer 40 may be etched into the bottom wall 14. It should also be appreciated that the masking layer 40 may be embodied in any given color which fits the needs of a given design of the sterilization tray 12.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A surgical instrument sterilization tray, comprising:
a bottom wall having an upper surface and a lower surface,
a plurality of sidewalls extending upwardly from the bottom wall so as to cooperate with the bottom wall to define an instrument storage surface area,
a plurality of instrument retainers secured to the bottom wall and configured to retain orthopaedic surgical instruments during sterilization and transport thereof, and
a masking layer disposed on the upper surface of the bottom wall within the instrument storage area, wherein the masking layer (i) defines a disruptive pattern comprising a plurality of connected common geometric shapes, and (ii) has a pattern coverage ratio of at least 50:50.

2. The surgical instrument sterilization tray of claim 1, wherein the masking layer is printed onto the upper surface of the bottom wall.

3. The surgical instrument sterilization tray of claim 1, wherein the masking layer is etched into the upper surface of the bottom wall.

4. The surgical instrument sterilization tray of claim 1, wherein the masking layer has a pattern coverage ratio of at least 60:40.

5. The surgical instrument sterilization tray of claim 1, wherein the bottom wall has a plurality of fluid holes formed therein.

6. The surgical instrument sterilization tray of claim 1, wherein the common geometric shape is a circle.

7. The surgical instrument sterilization tray of claim 1, wherein the common geometric shape is a hexagon.

8. The surgical instrument sterilization tray of claim 1, wherein the bottom wall and the plurality of sidewalls are constructed of anodized aluminum.

9. An orthopaedic surgical instrument system, comprising:
a surgical instrument sterilization tray constructed of anodized aluminum, the sterilization tray comprises (i) a bottom wall having an upper surface and a lower surface, and (ii) a plurality of sidewalls extending upwardly from the bottom wall so as to cooperate with the bottom wall to define an instrument storage surface area,
a plurality of instrument retainers secured to the bottom wall of the sterilization tray and configured to retain orthopaedic surgical instruments during sterilization and transport thereof, and
a masking layer disposed on the upper surface of the bottom wall within the instrument storage area, wherein the masking layer (i) defines a disruptive pattern comprising a plurality of connected common geometric shapes, and (ii) has a pattern coverage ratio of at least 50:50.

10. The surgical instrument system of claim 9, wherein the masking layer is printed onto the upper surface of the bottom wall of the sterilization tray.

11. The surgical instrument system of claim 9, wherein the masking layer is etched into the upper surface of the bottom wall of the sterilization tray.

12. The surgical instrument system of claim 9, wherein the masking layer has a pattern coverage ratio of at least 60:40.

13. The surgical instrument system of claim 9, wherein the bottom wall of the sterilization tray has a plurality of fluid holes formed therein.

14. The surgical instrument system of claim 9, wherein the common geometric shape is a circle.

15. The surgical instrument system of claim 9, wherein the common geometric shape is a hexagon.

16. An orthopaedic surgical instrument system, comprising:
a surgical instrument sterilization tray constructed of anodized aluminum, the sterilization tray comprises (i) a bottom wall having an upper surface, a lower surface, and a plurality of fluid holes formed therein, wherein each of the plurality of fluid holes extends from the upper surface to the lower surface, and (ii) a plurality of sidewalls extending upwardly from the bottom wall so as to cooperate with the bottom wall to define an instrument storage surface area,
a plurality of instrument retainers secured to the bottom wall of the sterilization tray and configured to retain orthopaedic surgical instruments during sterilization and transport thereof, and
a masking layer disposed on the upper surface of the bottom wall within the instrument storage area, wherein the masking layer (i) defines a disruptive pattern comprising a plurality of connected common geometric shapes, and (ii) has a pattern coverage ratio of at least 60:40.

17. The surgical instrument system of claim 16, wherein the masking layer is printed onto the upper surface of the bottom wall of the sterilization tray.

18. The surgical instrument system of claim 16, wherein the masking layer is etched into the upper surface of the bottom wall of the sterilization tray.

19. The surgical instrument system of claim 16, wherein the common geometric shape is a circle.

20. The surgical instrument system of claim 16, wherein the common geometric shape is a hexagon.
